(19) United States
(12) Patent Application Publication
(10) Pub. No.: US 2004/0055359 A1
(43) Pub. Date: Mar. 25, 2004

(54) Automatic gas sensor calibration system

(75) Inventors: Albert E. Ketler, Murrysville, PA (US); Bradley H. Baker, Pittsburgh, PA (US); Thayananthan Narayanan, Pittsburgh, PA (US); Lauren E. Sargent, Trafford, PA (US); Ronald C. Smathers, Ford City, PA (US); Ehren R. Graber, Monroeville, PA (US)

Correspondence Address: BROWDY AND NEIMARK, P.L.L.C., 624 NINTH STREET, NW, SUITE 300, WASHINGTON, DC, 20001-5303, US

(73) Assignee: REL-TEK, Monroeville, PA

(21) Appl. No.: 10/640,384

(22) Filed: Aug. 14, 2003

Related U.S. Application Data
(63) Continuation-in-part of application No. 09/893,343, filed on Jun. 28, 2001.

(51) Int. Cl. G01N 33/00; G01N 37/00

(52) U.S. Cl. 73/1.07; 702/100

(57) ABSTRACT

An automatic system for calibrating gas sensors comprising a source of SPAN gas and a source of ZERO gas, and a means to control the flow of these gases to sensors. The system schedules and makes all required sensor value corrections and adjustments without human intervention. High resolution monitoring enables life extensions for the sensors, as well as higher accuracy monitoring than would be reasonably expected from low cost sensors.

### CROSS-REFERENCE TO RELATED APPLICATIONS

The present application is a continuation in part of Ser. No. 09/893,343, filed Jun. 28, 2001, the entire contents of which are hereby incorporated by reference.

### FIELD OF THE INVENTION

The present invention relates to a method and apparatus for automatically calibrating gas sensors.

### BACKGROUND OF THE INVENTION

Gas detectors protect life and property. In an industrial setting, gas detectors typically use remote sensors so that the presence of any gas which may be hazardous, flammable, toxic, or otherwise important may quickly be detected at a remote location of a facility or process. The presence and concentration level of these particular gases can be monitored and electronically reported to a control room. The concentration of gas is typically analyzed by computer, and alarms are automatically activated when the concentration of a gas exceeds certain preset values. FIG. 1 shows a typical gas sensor array installed in a municipal bus maintenance garage where natural gas (methane) and carbon monoxide are monitored to prevent explosive and toxic hazards. Natural gas sensors N and carbon monoxide sensors C are dispersed throughout the garage. In this array, three automatic calibration stations AC are located, a subject of this invention. The natural gas sensors N are located in hazardous areas, while the carbon monoxide sensors C are not, thus requiring separation of the wiring conduits.

A major problem in any gas detection and alarm system is reliability. For the detection and alarm system to be dependable and safe, the operator must frequently check and re-calibrate each sensor. A large number of sensors are typically installed on a gas detection site, so that calibration is an ongoing, time consuming process. Because sensors for gases which are lighter than air, such as methane, ethane, hydrogen, etc. are sited high above the floor or ground, e.g., beneath the roofs of garages, inside skylights, inside silos, etc., applying calibration gas to these sensors and approaching these sensors to adjust the scaling can be hazardous to maintenance personnel. Similarly, the monitoring of heavy gases such as propane and gasoline, etc., often finds sensors installed in low pits and wells, again out of easy reach for calibration.

One early method for reducing the time, effort and risk of calibrating sensors, which is usually performed monthly, was by “remote calibration,” still a time consuming, manual procedure. This entailed installing long, ⅛ inch internal diameter tubes for delivering calibration gas directly to each sensor, using an adapter to direct the gas into the sensing head. Plastic tubing from each sensor led down to conveniently located calibration stations, usually mounted at eye level adjacent to the monitoring box. This type of monitoring box is described in Ketler, U.S. Pat. No. 6,169,488, the entire contents of which are hereby incorporated by reference. This monitoring panel is also referred to as a DXcalibar box. As many as 16 tubes, connected to as many sensors, were organized into a terminal manifold bracket, such that each tube was labeled as to the particular sensor it served. Each tube connection also had its own hose barb adapter for easily applying calibration gas from portable ZERO and SPAN gas tanks to any selected sensor via a flexible hose leading to the calibration apparatus, which included the source of the ZERO and SPAN calibration gas.

This old style manual/remote calibration process comprised applying ZERO gas (pure air) for about two minutes. The gas application time varied, depending upon the length of tubing, i.e., separation, between the sensor and the remote calibration station. The gas flowed through the tubing toward the sensors at about 100 feet per minute. A multimeter was used to monitor the analog electrical 4-20 ma signal from the sensor, which was accessible inside the attending DXcalibar box. When the ZERO setting became visibly stable, i.e., not changing with time, the technician adjusted the ZERO setting on the remote calibration circuitry, also within the DXcalibar box, so that the precise ZERO signal level, normally 4.0 ma, was viewed on a hand-held multi-meter. The ZERO gas was turned off by a hand valve and then SPAN (upscale) gas from the adjacent tank was applied for about two minutes. Again, when the sensor stabilized at the high level condition, the SPAN adjustment was made so that the signal read the particular gas concentration (e.g., 25 ppm carbon monoxide) as designated by the calibration certificate supplied with the calibration gas tank, presumably with accuracy traceable to the National Bureau of Standards.

Remote calibration, using tubes to route calibration gas to sensors, offered major benefits in reduced time for calibration and providing safer working conditions for personnel. However, this process still required the attention of a trained technician, and there was the possibility of errors occurring, such as if the gas flow timing was cut short by a careless technician, or if the meter readings and adjustments were imprecise. Indeed, if the adjustments were made prematurely, i.e., before the sensor signal stabilized, the result could be improperly adjusted, inaccurate sensors, which could not be relied upon to detect the presence of life threatening gas.

Traditionally, assuming 15 minutes of labor for manually calibrating only one sensor, a facility with 100 sensors could require 300 worker-hours each year for monthly calibrations. Assuming a typical labor cost of $40.00 per hour, the annual labor cost of calibration the 100 sensors would be about $12,000.00.

### SUMMARY OF THE INVENTION

It is an object of the present invention to overcome the aforesaid deficiencies in the prior art.

It is another object of the present invention to provide a method and apparatus for automatically calibrating gas sensors.

It is a further object of the present invention to extend the life of gas sensors.

It is yet another object of the present invention to provide two types of automatic gas sensor calibration (AGSC) systems which automatically make adjustments to the ZERO and SPAN gas sensor signals using high resolution computer techniques.

Two embodiments of AGSC systems of the present invention are described, each of which automatically adjusts the ZERO and SPAN values of gas sensors. The two embodiments described herein are referred to as (1) the Central Computer Telemetry (CCT) based system and (2) the Stand-alone Controller (SAC) based system, both of which produce similar AGSC results. The CCT requires that a central computer be available to control the AGSC procedure and record the data, while the SAC does not require a central computer but, instead, has its own on-board intelligence for managing the AGSC process totally independent of any other monitoring facility that may be present.

### BRIEF DESCRIPTION OF THE DRAWINGS

FIG. 1 shows a typical gas monitoring system installed in a municipal bus maintenance garage in which natural gas and carbon monoxide are monitored for explosive and toxic hazards, including three automatic calibration panels.

FIG. 2 shows the typical components of an overall CCT type gas monitoring system as applied to a facility similar to that shown in FIG. 1.

FIG. 3 illustrates an AGSC with pneumatic interconnections to the sensors and the calibration gas source tanks.

FIG. 4 is an exploded view of FIG. 3.

FIG. 5 illustrates a smaller, wall-mounted panel version of the system shown in FIG. 3.

FIG. 6 illustrates installation of calibration gas flow regulators and distribution manifolds throughout a large array of sensors to assure adequate calibration gas delivery to each sensor.

FIG. 7 illustrates a computer display form, wherein one or more AGSC sensor calibration zones can be set up and configured.

FIG. 8 shows a roll-around version of the CCT version.

FIG. 9 illustrates pneumatic quick-connections to the gas distribution manifold for a roll-around version.

FIG. 10 shows a computer display of an actual AGSC procedure for 8 sensors.

FIG. 11 shows a stand-alone controller, SAC version for 1-16 sensors.

FIG. 12 shows a DXcalibar box containing one SAC master card and two SAC slave cards for 8 sensors.

FIG. 13 illustrates how the present invention extends the useful life of gas sensors.

FIG. 14 illustrates a report generator showing results of various calibrations of a particular sensor. FIG. 14A is a bar graph showing calibrations 1-6, and FIG. 14B is a table showing the values obtained for calibrations 1-6.

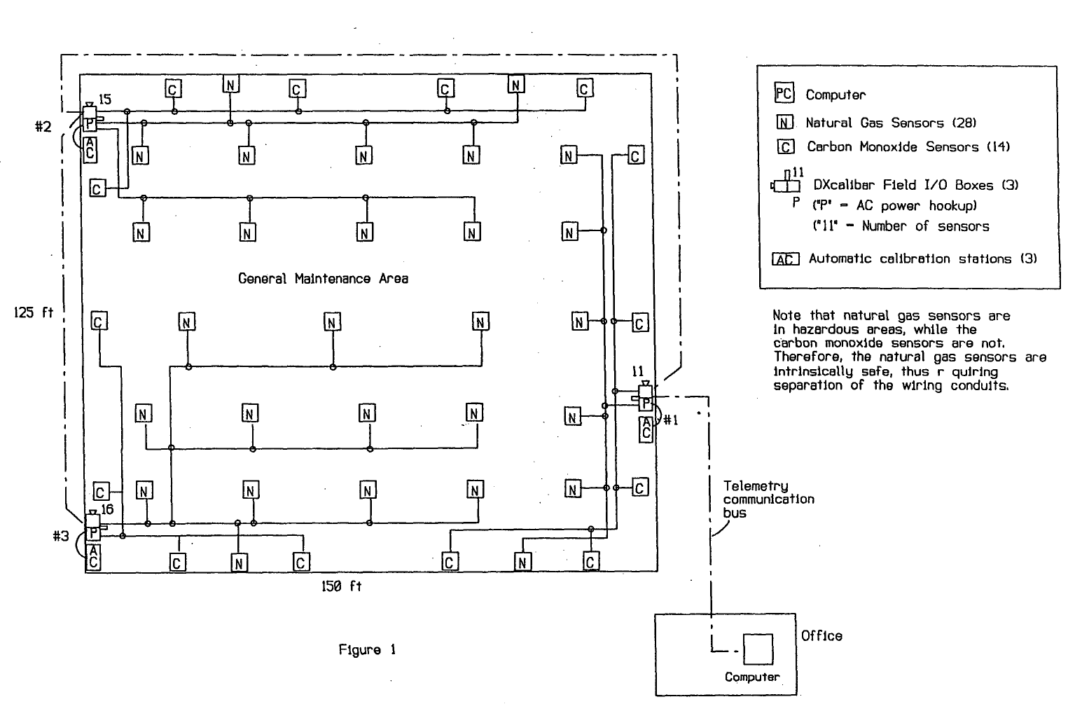

### DETAILED DESCRIPTION OF THE INVENTION

The Automatic Gas Sensor Calibration (AGSC) of large sensor arrays, typically 10 to 100 gas sensors, uses extensive networks of flow tubing and regulators, thus permitting full utilization of a single set of large, economical, calibration gas tanks (typically 6500 liters gas capacity compressed to 2500 psi), which can sustain the AGSC operations for a year or more.

The Computer Controlled System (CCT) of the present invention provides computer managed calibration using computer based software which controls and monitors through remote calibration stations. The system can be programmed to automatically calibrate sensors by time and date; by day of week and time; by day of month, day, and time; or on demand using a manual switch or mouse click which instructs the system to calibrate now.

The application of calibration gas is timed to account for gas travel time and speed of response of the sensors. The gas flow can be terminated by the computer based derived stabilization of the signal while the calibration gas is applied. Additionally, the computer monitors the calibration gas pressure to ensure an adequate supply of calibration gas.

The system provides a graphical setup form to organize the calibration parameters. The calibration sequences are timed to allow for the worst case flow rates and sensor responses. The sensors are calibrated within the computer files, not within the sensor itself.

The sensor signal range is set as a small part of the available computer range to allow for drifting up and down. In order to keep the tubing clear, it is preferred to apply the SPAN gas first, and then the ZERO gas. The computer regulates the SPAN and ZERO gas flow to produce uniform calibration gas sensor responses. A tubing matrix distributes calibration gas to multiple groups of sensors. Flow and pressure regulators compensate for differing flow demands, tubing resistances, and distances from the calibration gas supply. The alarms are automatically deactivated during calibration, and are reinstated upon conclusion of the calibration process. This system permits precision calibration using high frequency (e.g., daily or hourly) calibration with precision gases. The sensor calibration gas flow adapter overrides ambient gas monitoring during calibration events. The use of high resolution digitizing in the computer and data acquisition system extends the useful life of aging sensors.

Automatic gas sensor calibration is controlled by software resident in a central PC using a remote, multi-drop telemetry panel. This makes it possible to extend the scope of automatic gas calibration to large (i.e., 100+) sensor arrays using digital telemetry with multiple remote panels. The system provides graphical and text displays of historical records of calibration events and adjustments for each sensor. The system also includes telemetry to one or more DXcaliber boxes and remote control panels which incorporate automatic calibration gas solenoid valves and controls. This telemetry for remote panels controls the calibration gas and receives push button signals. These remote gas supply panels with solenoid valves, pressure monitoring, and indicators, are used for interfacing with remote field gas sensors. Graphical calibration reports convey sensor trends and impending problems.

Large, high pressure calibration tanks are used for handling large arrays of sensors. In this embodiment, two stationary tanks are chained or otherwise retained in location, incorporating high to low pressure regulators and pressure transducers. Pressure transducers are monitored through telemetry channels to provide the user with information on gas inventory and possible leaks, as well. All electrical interconnections are preferably by quick plugs and push on connectors for speed and accuracy of maintenance.

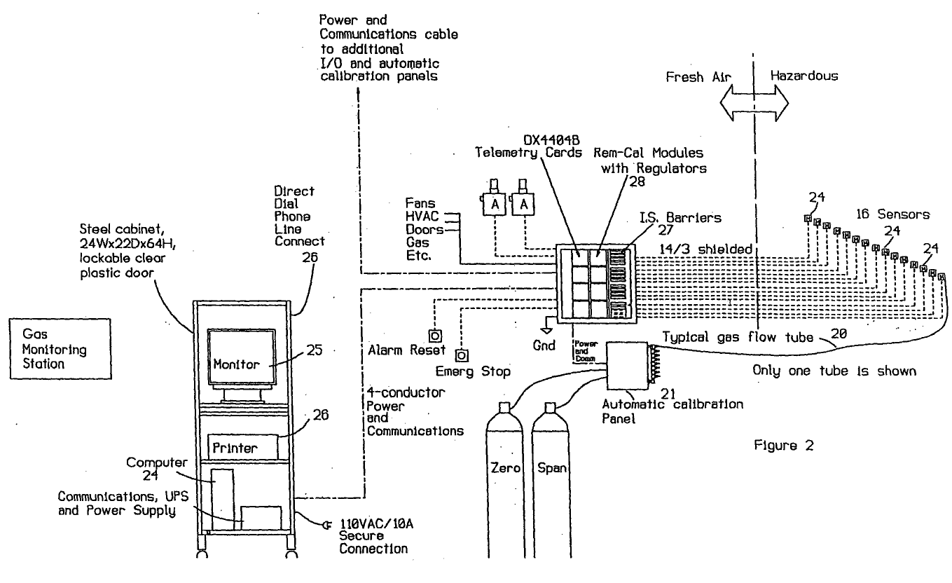

FIG. 2 illustrates the typical components of a CCT overall gas monitoring system, including a typical gas flow tube, an automatic calibration panel, SPAN gas tank 22 and ZERO gas tank 23. In this illustration 16 sensors 24 are connected to the system.

The gas monitoring station includes a computer 24, a printer 26 and a monitor 25. There is a direct dial phone line connect 26. Between the sensors 24 and the gas monitoring station are intrinsic safety barriers 27 and a current regulator and adjustment module 28.

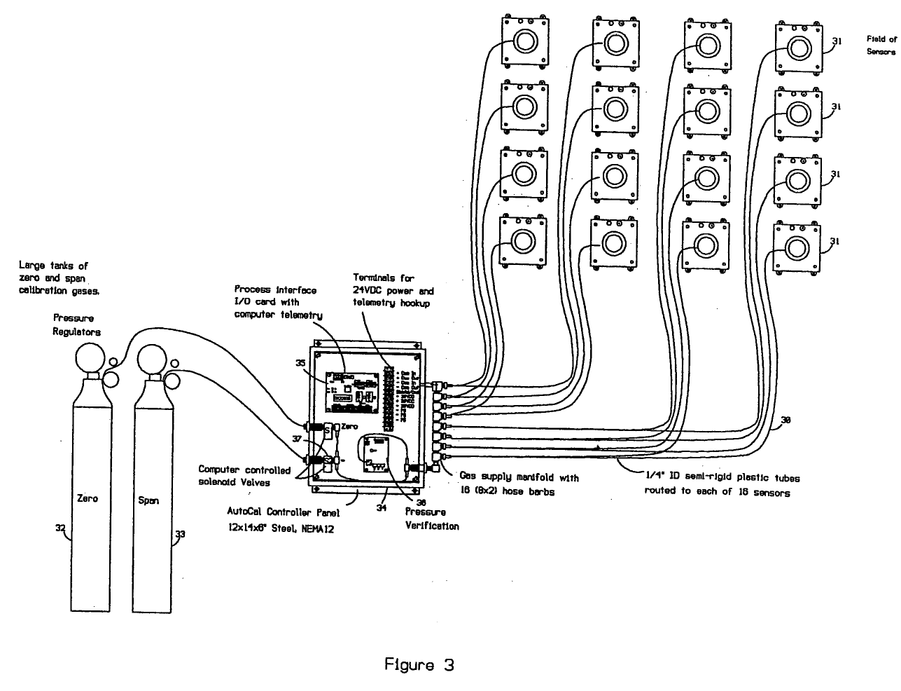

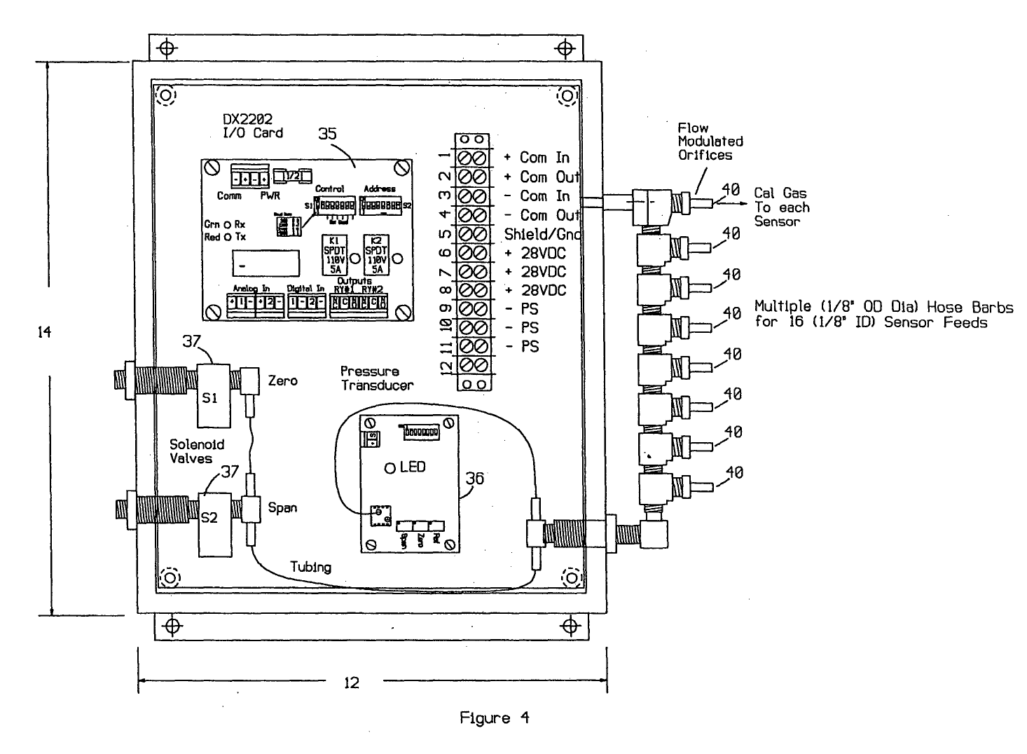

FIG. 3 illustrates the CCT-AGSC with its pneumatic interconnections 30 to the sensors 31, and the cal-gas source tanks, ZERO 32, SPAN 33. This figure also shows the wall mounted control panel 34 containing the telemetry control card 35 for communicating with the computer, the solenoid valves 37 which control the cal-gas flow, and a pressure transducer 36 for monitoring the cal-gas supply pressure to assure adequacy during the GASC procedure. Details of this system are better seen in the exploded view of FIG. 4, which better illustrates the flow regulators 40 which send calibration gas to each sensor.

The AGSC system of the CCT type, shown in FIGS. 3, 4, and 5, provides archival documentation of the periodic AGSC events within the central computer, including dates and times and logs of all sensor values before and after calibration. Summary reports can be automatically printed and stored for archival retrieval, as well as for off-line verification and analysis.

Pre-programmed AGSC scheduling and timing permits precisely conducted AGSC procedures to avoid government fines and citations arising from forgotten or improperly conducted sensor calibration. This AGSC can save the costs of fees and, more importantly, provides additional safety to the area in which they are installed.

AGSC avoids the high labor costs and possible personnel safety compromises attending manual sensor calibration. This is especially important when sensors are located in hazardous areas such as sealed sections of coal mines, storage silos, roadway tunnels, over machinery, in pits or wells, or in elevated or other hard to reach places requiring ladders and lifts.

AGSC provides a high degree of accuracy and consistency in calibration that is often absent with manual calibration, including human errors resulting from careless gas flow timing, uncertain sensor stabilization recognition, gas flow rate adjustments, uncertain meter accuracies and calibrations, meter readout interpretations, clumsy screwdriver adjustments, and the like.

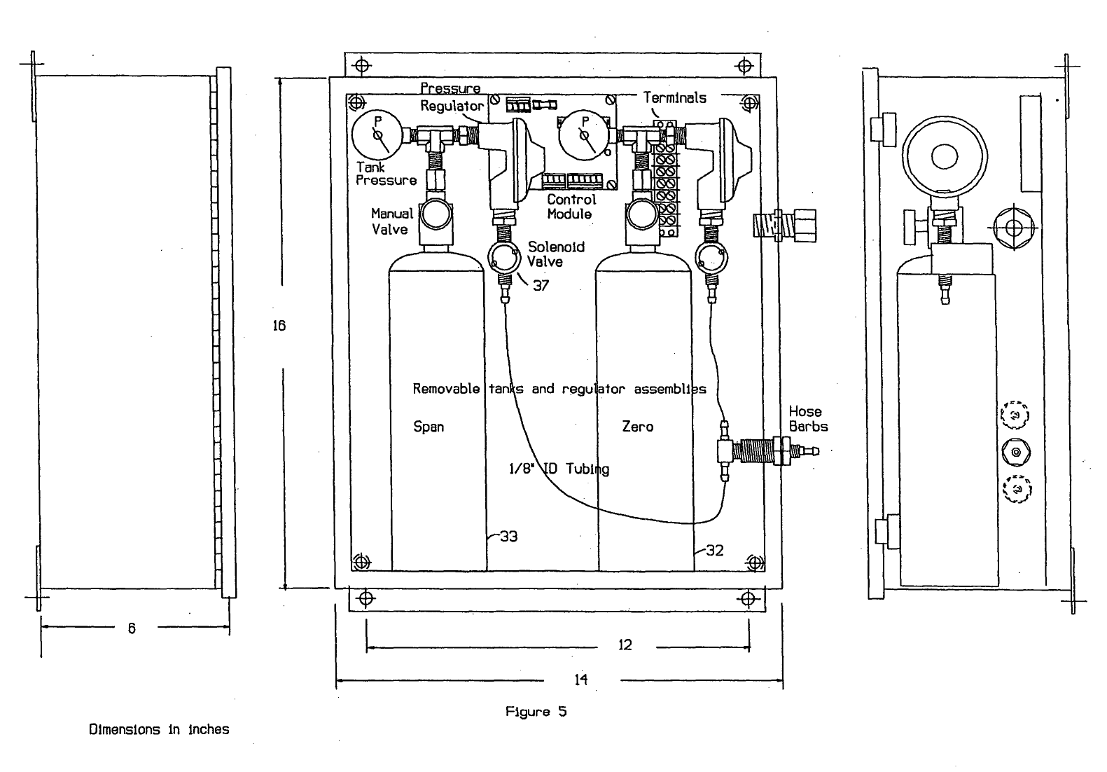

The small version of the CCT concept, illustrated in FIG. 5, provides a more compact format for providing AGSC for just a few sensors, where large supplies of calibration gas are not needed. In this case, 15-liter, 250 psi tanks measuring just three inches in diameter by 18 inches in height are conveniently packaged inside a wall mounted panel along with the electronics, pressure transducer, and solenoid valves.

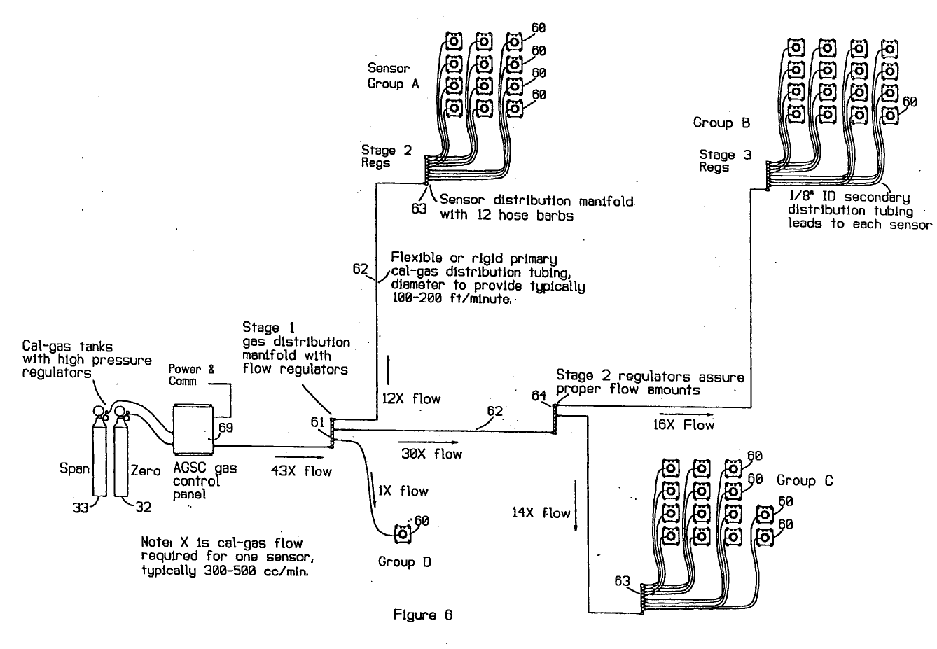

Referring to FIG. 6, uniform distribution of calibration gas to a large array of sensors 60 which may encompass a 1000 ft radius, was a problem that was solved by installing gas flow regulators and distribution manifolds throughout the array, as shown in FIG. 6.

FIG. 6 shows SPAN 33 and ZERO 32 gas tanks connected through a pressure regulator 36 to a gas control panel 69. In the first stage, there is a gas distribution manifold with flow regulators 61. Distribution tubing 62 leads to a sensor distribution manifold 63 which distributes SPAN and ZERO gas to the sensors 60. Stage 2 regulators 64 assure proper flow amounts to other groups of sensors 60. The concept ideally permits all sensors to reach stabilization simultaneously to avoid gas wastage and minimizing calibration time.

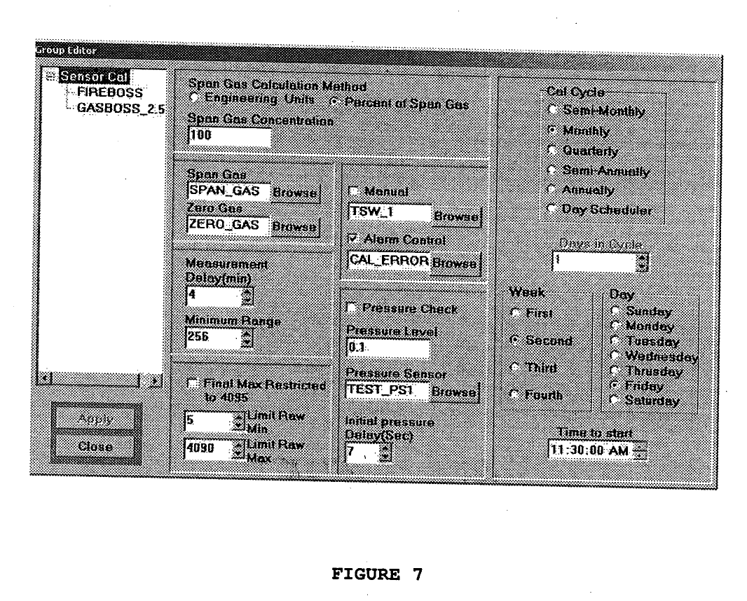

FIG. 7 illustrates the computer display wherein an AGSC zone can be set up and configured. Sensors assigned to the zone are selected. The calibration dates and times are entered, as well as the duration of flow of the calibration gas and the minimum cal-gas pressure permitted during the procedure. All connected alarms are also designated, so they can be automatically disabled during the AGSC procedure.

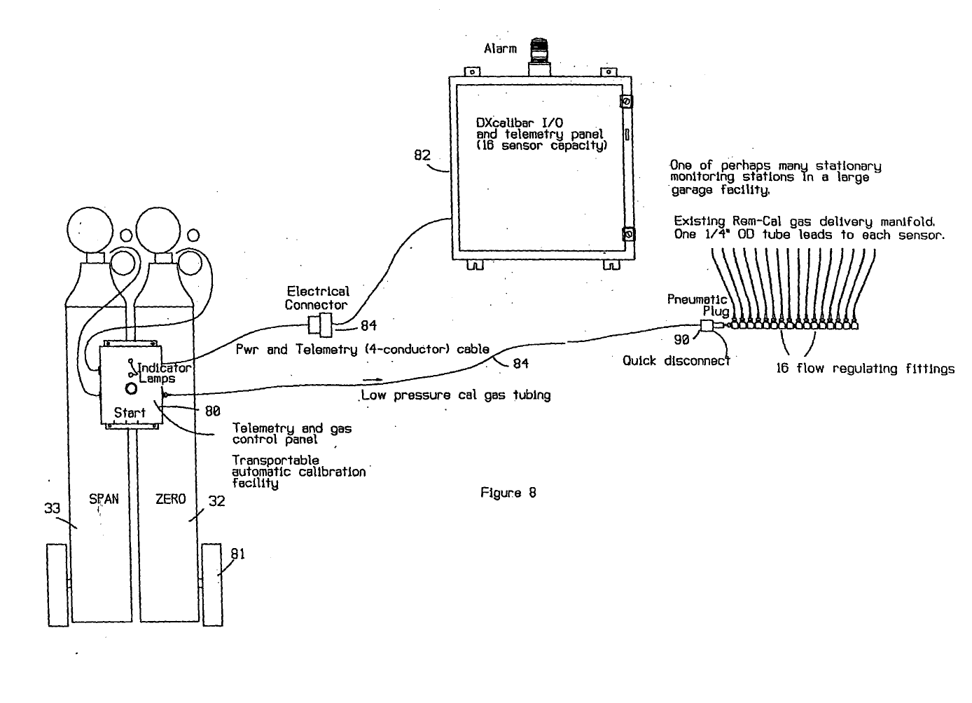

FIG. 8 shows a roll-around version of the CCT embodiment wherein the usually stationary cal-gas tanks 32, 33 and control panel 80 are installed on a hand truck 81 which can be moved to multiple zones. Each of numerous I/O panels 82, usually DXcalibar boxes, is already fitted with sensors with cal-gas tubing 84 and a hose barb manifold 61. The operator wheels the mobile AGSC apparatus in place and connects to the zone controller with a pneumatic plug 90 and an electrical connector 84. The central computer immediately recognizes the I/O card address of the mobile unit and the zone where it is connected. The operator presses a “Cal-Now” button on the mobile panel and the computer starts the AGSC procedure within that zone. The benefit of the roll-around embodiment is that it reduces the capital investment of providing a stationary AGSC station at each of possibly 20 or more zones. It does, however, require some attention from an operator to move and connect the mobile apparatus among the zones, thereby offsetting some of the labor savings benefits of AGSC. With this embodiment there is also the slight possibility of error from, e.g., the operator forgetting to calibrate a zone, bad pneumatic or electrical connections, leaky fittings, or the like.

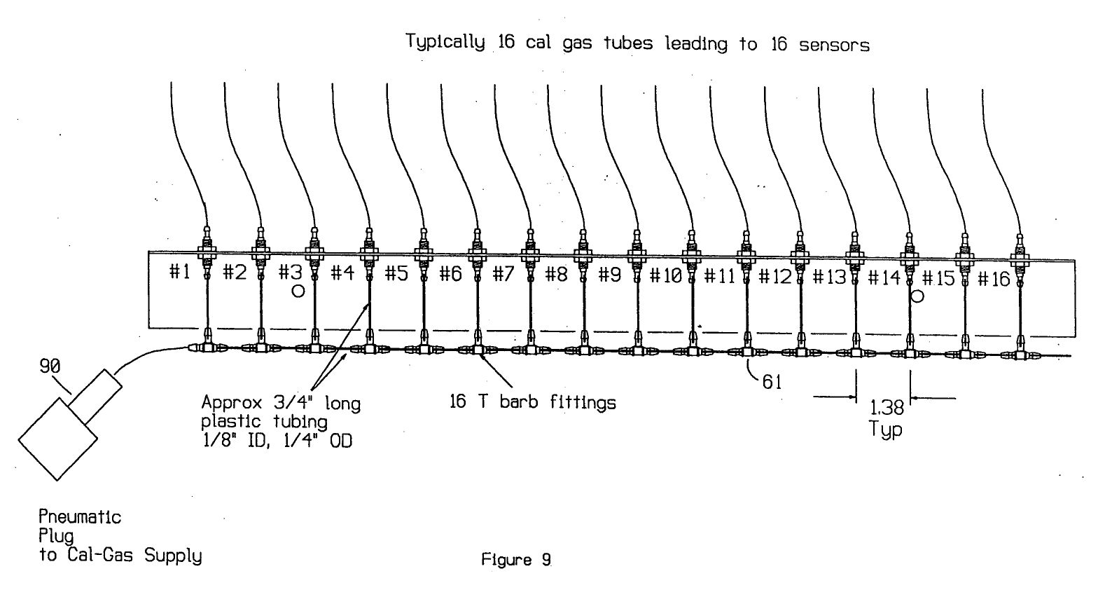

FIG. 9 illustrates the pneumatic quick-connections to the gas distribution manifold 61 which are preinstalled in each zone.

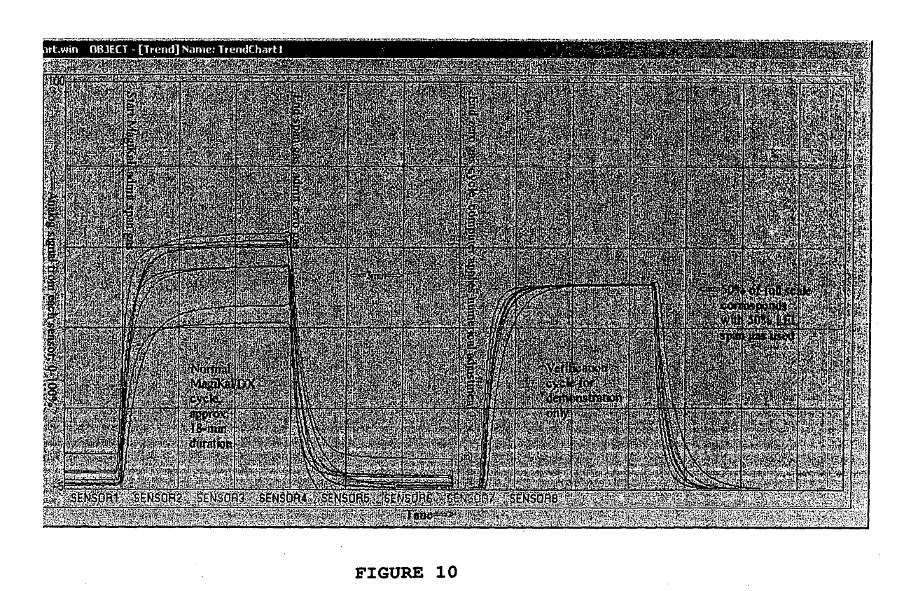

FIG. 10 shows a computer display of an AGSC procedure. In this instance, eight methane sensors were automatically calibrated as a group (or zone). Viewing from the left of the chart, SPAN gas was applied for several minutes as the gas flowed to the sensors and registered with up-scale readings. The maximum, stable upscale values were recorded by the computer, and the computer then issued commands to turn off the up-scale gas flow and turn on the ZERO gas flow. Waiting for the prescribed stabilization time, the computer then registered the ZERO signals in memory and terminated the gas flow. Immediately after this test termination, the new SPAN and ZERO signal parameters were applied to the sensor calibration registers within the computer memory, providing perfectly calibrated sensors which can be depended upon for critical measurements. To verify that the sensors were indeed calibrated properly, the test was rerun, admitting SPAN and ZERO gas as before, as shown on the right side of the chart, with upscale and ZERO values conforming precisely to the calibration gas mixtures.

AGSC requires at the minimum a calibration gas distribution network for distributing the calibration gas to the sensors during the AGSC, illustrated in FIG. 6. The systems use an array of tubing, in conjunction with pressure gradients and multiple flow regulators, which together

a. minimize the time for gas to flow to the farthest sensors;

b. assure adequate calibration gas flow to each sensor, regardless of the distance;

c. permit longer flow paths within reasonable time constraints; and

d. minimize the consumption of valuable calibration gas.

Multiple component gas mixtures (e.g., 2.5% methane, 21% oxygen, 50 ppm CO, 1000 ppm CO₂, balance nitrogen) make it possible to simultaneously calibrate a mixture of sensor types on one gas distribution network using one set of calibration gas supply tanks.

In a preferred embodiment, a computer is programmed to schedule AGSC events, which are usually timed to occur at night, on weekends, or other off times, to minimize the impact of gas sensor downtime while offline during calibration. The central controlling computer maintains detailed logs of sensor values before and after the SPAN/ZERO calibration cycle, providing valuable historical information on the aging, drifting, and general performance of each sensor, triggering preventive maintenance guides for wary users. The computer software and telemetry automatically disable alarms to avoid nuisance and unnecessary alarm activation during the AGSC cycle. A setup choice can be introduced to permit alarms to activate normally while calibrating, as in the case of infrequent total system testing.

The CCT system automatically disables any sensor that fails to provide sufficient SPAN-ZERO signal movement, typically at least 0.8 ma during the AGSC cycle, thus providing an additional safety benefit. The computer simultaneously provides an urgent message to notify management of the need for special maintenance services required to replace the sensor and invoke another calibration.

A “Cal-Now” command forces the AGSC cycle to commence immediately to perform a full AGSC of all sensors in a designated group.

Testing for calibration gas SPAN/ZERO supply pressures before, during and after the AGSC cycle assures adequacy of gas for the procedure to be valid. Using customized, pre-set pressure regulators to reduce from the high pressure (e.g. 2500 psi) calibration gas tanks avoids the need for client adjustments and possible tampering that could over-pressure the control valves or cause the AGSC event to default for inadequate gas pressure.

Calibration software used with the present invention runs as a background task, timing automatic calibration and scheduling based on the software calendar and clock of the computer. When calibration time is at hand, the calibration software sends a message to the system which monitors a large number of gas sensor signals for alarm conditions, to disable all alarms for all sensors connected to the calibration panel which are to be calibrated. The calibration software communicates to the particular calibration panel at the field location near the sensors over a telemetry link, instructing the resident telemetry card to open the SPAN-gas solenoid valve. A pressure transducer at the remote panel monitors the gas pressure supply, the status of which is continuously telemetered to the computer. If the calibration gas pressure drops below a preset threshold, the calibration software aborts the AGSC procedure, returns sensors to prior values, and prints a message on the computer screen to notify the operator. If the pressure is sufficiently high, indicating ample gas supply, the procedure continues, with the upscale SPAN calibration gas (usually a gas concentration of 50% of the full scale range of the sensor) flowing through the manifold containing flow regulators, then through the connected tubing, arriving at each sensor, and then entering each sensor's gas detection module. Following a pre-set time duration, or whenever the farthest sensors are determined by the computer to have stabilized at the SPAN calibration gas value, the computer sets the new SPAN values in its digital calibration data base to precisely the value of the SPAN calibration gas concentration, for example, 15.2155 ma, within 12-bit resolution.

The computer then issues control commands to the calibration panel to shut off the SPAN solenoid and open the ZERO-gas solenoid valve. Again, the gas pressure is verified to be sufficient before proceeding. The ZERO-gas, which may be pure air or nitrogen having no upscale components, flows through the manifold of regulators and tubing, pushing ahead of it any SPAN-gas remaining in the tubing, and finally arriving at the sensor. After a timed period, or whenever the ZERO level is determined to be stable, the computer proceeds to set the ZERO values in its digital data base to the same 12-bit resolution, e.g. 4.2643 ma.

To terminate the calibration procedure, the calibration software program detects the condition and sends a command to the calibration panel to shut off the ZERO-gas solenoid valve. The computer then returns the newly calibrated sensors to their normal operational status, and any alarming functions are reactivated.

The computer generates a final report giving the results of the auto-calibration event, including the time and date, the SPAN and ZERO values for each sensor before and after gases are applied, and listing any sensors which tested badly. Failed sensors are automatically disabled (i.e., removed from gas monitoring service) and are flagged in the final report for special maintenance attention.

It is preferred that SPAN testing be performed prior to performing ZERO testing, as it is better to leave the gas supply tubing filled with ZERO-gas after testing than with upscale SPAN-gas. Leaving SPAN-gas in the gas supply tubing would require purging this tubing, which is a wasteful use of ZERO-gas. Performing SPAN testing first and ZERO testing last leaves the tubing nicely purged with ZERO-gas at the end of the test.

Timed periods for each gas component flow are typically about 1-5 minutes, although this depends on the length of calibration gas delivery tubing involved (approximately 100 ft per minute flow rate) and the response speed of the sensor under test. A complete calibration procedure with 200 foot sensor ranges can take about 8 minutes.

Calibration scheduling can be flexibly programmed to occur at any time of any calendar day. Multiple groups or zones can be identified to be processed at different times. A low-pressure threshold can be entered, as well as the wait time and gas concentration parameters. In the event the user wishes to run an unscheduled, immediate recalibration, such as after sensor replacement or an alarm incident, the “Cal-Now” button can be selected, which will start the procedure immediately.

The calibration gas manifold can be designed with flow regulators to provide nearly equal calibration gas flow to each sensor. Without these regulators, a tube with the least flow resistance would receive excess calibration gas, while a tube with a higher flow resistance would receive less gas flow. Since the gas flow and sensor output stabilizing time period should be ideally identical for all sensors, the sensor having the least flow may be inadequately stabilized at the end time when the sensor signal values are accepted. This would result in inaccurate calibration and possibly create an unsafe condition. Of course, the time period would be extended to assure ample stabilization time for the slowest, or highest resistance, path. This problem is solved by the invention by providing constant-flow regulators in critical distribution paths, such that the variable portion of the tubing resistance among sensors would be an insignificant variation. FIG. 6 illustrates this.

Instrumental in the present invention is a report generator, a software utility that runs on the CCT computer and can be called up on user command. This report generator produces and displays calibration records, which are resident in the computer's hard drive memory, in a concise, bar-graph format with tabulated data for easy review and interpretation. The report generator prompts the user on what is required to correct any problems. For example, if a sensor signal approaches or exceeds the high or low boundaries for proper digitizing, the display bar for that calibration date changes from green to blue to red, depending upon the severity. It also shows if the dynamic range of the sensor becomes too small to calibrate properly, and displays an instruction to service the sensor. In an instant, the report generator saves the user vast amounts of time that would otherwise be required to analyze the myriad calibration records and uncover any problems. The graphical imaging avoids the tedium of a trial-and-error approach that could result in improper sensor adjustments with possible safety consequences. The graphical and tubular summary reports can be printed for distribution and filing.

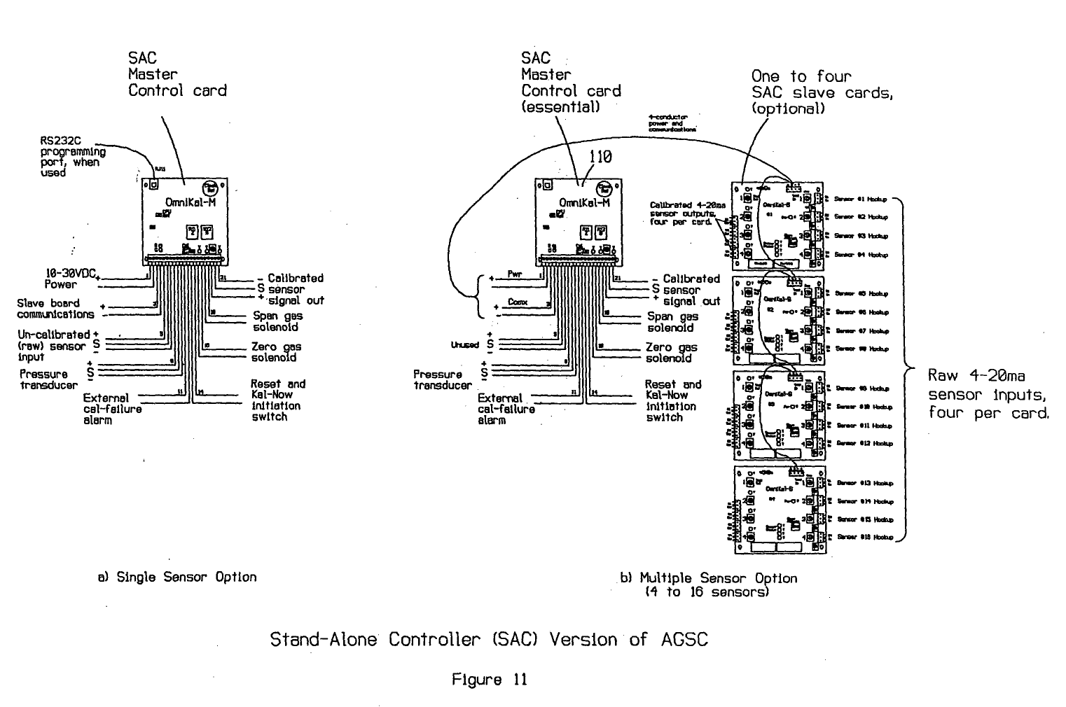

The Stand Alone Controller (SAC) version shown in FIG. 11 includes a “master card” with memory for controlling the storing of as many as 384 calibration dates, which is the capacity of the particular memory card used. This number will vary, of course, depending on the particular memory card used. For long-term, multi-year daily calibration, which would exceed the memory capacity of most currently available memory cards, the software includes a “daily” calibration set up option. The “master card” is programmed using a graphic format downloaded from a plug-in laptop or other type of PC. This master card can monitor one 4-20 ma signal from one sensor. Digital telemetry communicates with “slave cards” to expand the capacity of the system. Relays are used for controlling SPAN and calibration gas flows to the sensors during calibration. Error detection and fault indicators alert the user to calibration problems. This system includes means to supply the 4-20 ma signals which existed prior to calibration throughout the actual calibration event to avoid activating alarms.

Slave cards communicate with the master card to expand the sensor capacity for automatic calibration. Each slave card can handle four 4-20 ma sensor input signals, and to generate four calibrated output signals. The signal values are digitized and communicated to the master card during calibration, and at the end of the calibration procedure, the slave cards receive updated calibration values for the four sensors.

In the Stand Alone Controller (SAC) embodiment, the AGSC GASC benefits are extended to include alien sensors (i.e., those of other manufacturers). This embodiment, shown in FIG. 11, consists of a master control card 110 containing an onboard, stand-alone computer with all necessary code, a battery supported calendar/clock chip and inter-card telemetry. The SAC is inserted into the 4-20 ma signal and power cables already present between the gas sensors and the monitoring equipment (e.g. an alien computer, a chart recorder, a data logger or the like) that may be present. The master SAC card has the stand-alone processing capability for handling (i.e., calibrating) just one alien 4-20 ma gas sensor (see FIG. 11a). Using software and a setup screen similar to that shown in FIG. 7 installed in a portable laptop PC, all of the necessary setup information is downloaded to the SAC master card over a plug-in cable (shown in FIG. 11 as an RS232 serial port connection). After downloading the instructions and initializing the SAC computer memory, the portable PC is disconnected and removed from the area. The SAC card has two output relays for activating the SPAN and ZERO cal-gas solenoid valves as the scheduled AGSC process progresses. An alarm activation output relay is provided for alerting management if the AGSC process is not concluded successfully. While the AGSC procedure is underway, the pre-AGSC signal is sent out by the SAC master card, followed on completion of the AGSC cycle by the calibrated signal, all without interruption. After the AGSC procedure is successfully concluded, the precisely calibrated 4-20 ma signal is outputted to whatever monitoring equipment is present, such monitoring equipment being unaware of any intervening AGSC event occurring.

To expand this SAC-AGSC capability to service the calibration needs of multiple gas sensors, the present invention includes digital telemetry in the master card for communication with up to four SAC slave cards, each card having the capability of handling four gas sensor circuits. This is illustrated on the right side of FIG. 11. For the purpose of clarifying details of the invention, the sensors and monitoring equipment are not shown in the Figure. In this case, the master card schedules and controls the AGSC procedure, activates the solenoid valves, and controls the abort signal, all the while monitoring all individual sensor signals telemetered from the slave card or cards in turn. Update (polling) frequency to the slave cards is multiple times per second. When connected with one or more slave cards, the master card is unable to monitor its single sensor port, and this single sensor input-output channel is ignored. Switches on the slave cards identify the address of the slave (i.e., 1-4) so that the master card's configurational information (e.g., sensor name, type, location and channel) can be individually identified for each channel of the slave card array. Assuming the maximum of four slave cards, each with four connected alien sensors, the maximum sensor count for the system, one master card and four slave cards, is 16.

Each slave card has up to four 4-20 ma (uncalibrated) sensor input channels and a corresponding number of 4-20 ma (calibrated) signal outputs. During the AGSC procedure, the signals outputting immediately before the procedure continues to output unchanged during the procedure. Thus, there is no need to disconnect any threshold alarms or controls that would be otherwise activated as the upscale cal-gas flow is applied during the AGSC procedure.

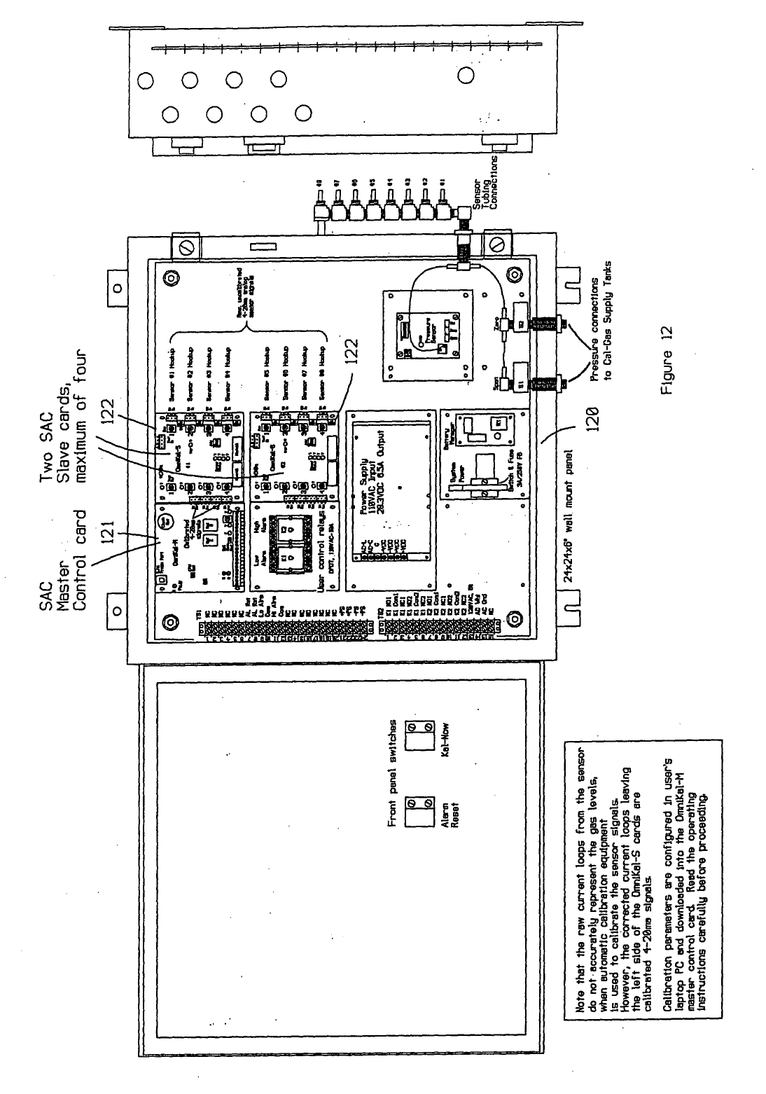

FIG. 12 shows a DXcalibar type box 120 containing one SAC master card 121 and two SAC slave cards 122, constituting a total AGSC capability for eight alien gas sensors. This stand-alone control panel contains a power supply, backup battery and all essential supporting components for providing AGSC functions.

When installed, the SAC input/output set is invisible to any existing monitoring equipment in place, as each output contains a calibrated 4-20 ma signal which is similar to the original 4-20 ma signal coming from the sensor, but calibrated.

In one embodiment of the SAC system, the CPU is contained on one SAC-M master card which contain the program code for scheduling and documenting each AGSC event. It also contains solenoid valve controls, as well as one set of 4-20 ma input/output ports for calibrating one gas sensor by itself when used alone, i.e., not connected to any salve cards. It also has a serial communication port for downloading setup information from a standard lap-top or other computer, as well as non-volatile memory chips for storing these instructions and the historical calibration data generated from the GASC cycles. An alarm relay is included for connection to an external alarm circuit for alerting management of any failure, lack of gas, or the like that could constitute a safety problem.

Blinking lights are provided on all PCB cards to show when communication is in progress. The master card controls the SPAN and ZERO gas flows, while the slave card(s) accept the sensor inputs and generate new calibrated outputs for each connected sensor. Setup of the calibration parameters in the auxiliary laptop or other computer includes the sensor type and range, the calibration dates and times, the SPAN calibration as concentration, digitizing values, and other information modeled after the CCT scheme described above.

Because every gas sensor has a drift rate, some higher than others, its accuracy and precision can be related to the time which elapsed since its last calibration. Of course, the calibration gas is the standard, being supplied to accuracy traceable to the National Bureau of Standards, thus establishing the upper limit accuracy for any calibration or gas detection process. So, for precise gas detection performance, it is essential that sensors be calibrated frequently, thus minimizing the opportunity for drifting and consequent detection errors. This AGSC invention permits frequent calibrations, daily or even hourly, so inexpensive, industrial grade sensors (i.e. those more prone to drifting) are able to perform with equal or better precision than higher priced, analytical grade sensors. The AGSC embodiment uses 12-bit analog-digital conversion, enabling calibration parameters to be discerned to within ±0.02% resolution. This is 50 times finer than the 1% analytical calibration gas mixture certifications that are generally available from cal-gas suppliers. Therefore, by calibrating frequently, the invention enables low cost sensors to perform in the same league as higher cost analytical instruments.

### Background of Gas Sensor Aging

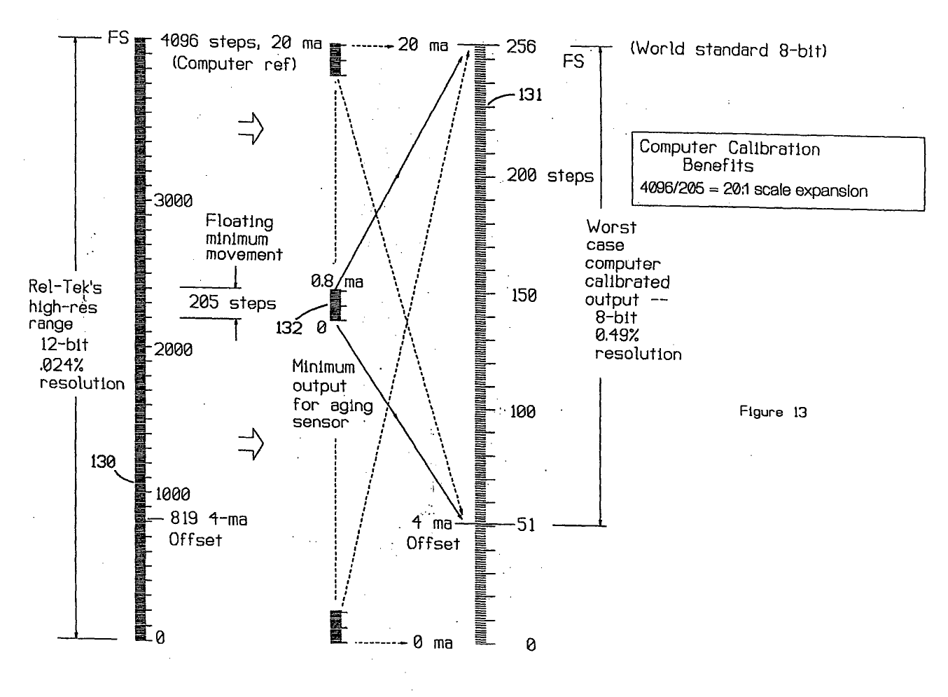

FIG. 13 illustrates how the AGSC of the present invention, in either embodiment, extends the useful life of the gas sensors. The following discussion presents salient information needed to understand the life extending capabilities of the system.

The aging phenomenon of typical gas sensors manifests itself in a reduction in the dynamic range available. Indeed, short of a catastrophic failure, the 4-ma ZERO level signal gradually creeps up and the 20 ma SPAN level signal gradually creeps down, thus diminishing the amount of signal change between min. and max. Sensors have ZERO and SPAN adjustments to compensate for this drifting, within reason, but a point in the aging process is eventually reached where the low setting can no longer be adjusted to read 4 ma and/or where the max setting can no longer read 20 ma, or whatever maximum signal is deemed appropriate. When the signal fails to adjust to the appropriate cal-gas application, it is usually deemed to have failed and the sensor is replaced, which is a costly and labor intensive procedure. There is great economic benefit if the reduced signal range of an aging sensor can be utilized.

The AGSC of the present invention uses a 12-bit digitizing resolution on the analog input and output channels. This is illustrated as a 4096 line scale 130 shown in FIG. 13. On the other hand, the majority of the world's monitoring systems use a coarser 8-bit resolution analog to digital conversion. This is illustrated on the less precise vertical scale 131 showing 256 digitizing steps. Ratioing these two dynamic ranges shows a 16:1 difference. The tiny bar 132 between these two outside ranges illustrates the worst case signal that can still be monitored and productively used to portray the ZERO-SPAN dynamic gas range at the computer in no less than 256-bit resolutions, which is satisfactory for most of the world's gas sensing and monitoring applications and which, for the purposes of the present invention, is used to identify the worst case for most practical monitoring situations.

In configuring the AGSC computer screens, illustrated in FIG. 7, there is a portion of the form requiring the entry of the minimum range (number of digitizing steps) deemed acceptable, i.e., the threshold below which a sensor is rejected as failing to calibrate properly during and AGSC procedure. The operator may choose the 8-bit, 256 step world standard, or any other range up to and including the 12-bit (4096 step) precision limitation of the equipment used in the embodiments of the present invention.

Thus, the life of an aging sensor may be extended for whatever extra time there may be available before the signal shrinks from the threshold 4-20 ma standard to the 0.8 ma minimum dynamic range associated with the present invention.

Similarly, there are higher resolution analog-to-digital conversion chips (e.g., 14 bit, 16 bit, etc.) and compatible microprocessors commercially available that can provide even higher dramatic range ratios, enabling the minimum usable signal range to shrink even further than 0.8 ma, thus extending the useful lives of valuable but aging gas sensors even longer. The embodiments of the present invention are not intended to be limited by the 12-bit resolution components disclosed in the embodiments described, but encompass the use of other higher resolution components that make the life extending benefits of the present invention even more pronounced.

While the examples in the present specification are illustrated with a 4-20 ma signal, the present invention is not limited to this type of signal. The present invention for automatic gas sensor calibration can be used with other signal modes, including but not limited to voltage, digital, and the like.

The foregoing description of the specific embodiments will so fully reveal the general nature of the invention that one can, by applying current knowledge, readily modify and/or adapt for various applications such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

What is claimed is:

1. A process for automatically calibrating gas sensors comprising:
a. connecting a calibrating gas tube to each sensor;
b. introducing SPAN or ZERO gas into a sensor calibration tube manifold;
c. waiting a predetermined time for the SPAN or ZERO gas to travel to each sensor and/or for the sensors to register the SPAN gas in a stable fashion;
d. detecting the raw digital value transmitted for each sensor and assigning calibrated values to the proper sensor configuration registers;
e. terminating the flow of SPAN or ZERO gas;
f. introducing ZERO or SPAN gas into the sensor calibration tube manifold wherein both ZERO gas and SPAN gas are used;
g. waiting a predetermined time for the ZERO or SPAN gas to travel to each sensor and/or for the sensors to register the ZERO or SPAN gas in a stable fashion;
h. assigning calibrated values to the sensor registers; and
i. turning off the ZERO or SPAN gas flow.

2. The process according to claim 1 wherein computer based software controls and monitors the calibration process.

3. The process according to claim 2 wherein calibration events are automatically scheduled.

4. The process according to claim 2 wherein the application of calibration gas is timed and/or monitored to account for gas travel time and speed of sensor response.

5. The process according to claim 1 wherein SPAN gas is applied first, and ZERO gas is applied second.

6. The process according to claim 2 wherein the flow of SPAN and ZERO gases is regulated to produce uniform calibration-gas sensor response.

7. The process according to claim 2 wherein the flow of sensor gas is adapted to override ambient gas monitoring by the sensor during calibration.

8. The process according to claim 2 wherein high resolution digitizing is used to extend the useful life of the sensors.

9. The process according to claim 1 wherein the flow of ZERO gas and SPAN gas is controlled by computer-controlled solenoid valves.

10. The process according to claim 1 wherein each sensor includes permanently installed tubing and fittings which direct any calibration gas flowing through the tube directly into a sensing head of the sensor.

11. The process according to claim 2 wherein digital telemetry with remote panels is provided.

12. The process according to claim 2 further providing graphical and text displays of historical records of calibration events and recommendable adjustments for each sensor.

13. The process according to claim 2 further comprising telemetry to remote panels for controlling calibration gas and receiving signals.

14. The process according to claim 2 wherein cal-gas supply pressures are monitored through telemetry channels.

15. The process according to claim 1 wherein the calibration dates and times are calculated and/or stored on a master card in a stand-alone system.

16. The process according to claim 15 wherein slave I/O cards communicate with the master card.

17. Apparatus for automatically calibrating gas sensors comprising:
a. a calibration gas tube connected to each sensor;
b. a source of SPAN gas and a source of ZERO gas;
c. a computer to control introduction and termination of SPAN gas and ZERO gas to each sensor, wherein said computer directs performance of automatic gas calibration on a scheduled basis.

18. The apparatus according to claim 17 wherein multiple types of sensors are included in the group of sensors, and calibration gas mixture contains various precisely quantified amounts of each gas component for calibrating each type of sensor in the group in unison.

19. The apparatus according to claim 17 wherein each sensor includes a permanently installed adapted fitting which directs any calibration gas flowing through the tube directly into the sensing head of the sensor.

20. The apparatus according to claim 17 further including solenoid valves for introducing the SPAN gas and the ZERO gas.

21. The apparatus according to claim 17 wherein the apparatus is stand-alone and transparently adaptable to application on “alien” sensor systems.

22. The apparatus according to claim 17 further comprising a master card containing a microprocessor, memory, solenoid valve drivers, and a clock and/or calendar chip.

23. The apparatus according to claim 23 further comprising at least one slave card connected to the master card, said at least one slave card containing up to four 4-20 ma (or other) raw signal inputs and with corresponding 4-20 ma (or other) calibrated signal outputs.

24. The apparatus according to claim 17 wherein intrinsic safety barriers are provided between the sensors and the gas monitoring station.

25. The apparatus according to claim 17 wherein the automatic gas sensor calibration system has interconnections to the sensors which are pneumatic, electrical, or a combination of pneumatic and electrical interconnections.
